(19) United States
(12) Patent Application Publication
(10) Pub. No.: US 2013/0059294 A1
(43) Pub. Date: Mar. 7, 2013

(54) IDENTIFICATION OF POLYMORPHIC HEPATITIS B VIRUSES AND KRAS ONCOGENE MUTATIONS AND CLINICAL USE

(71) Applicants: Xiangdong Ren, Malvern, PA (US); Alec S. Ren, Malvern, PA (US)

(72) Inventors: Xiangdong Ren, Malvern, PA (US); Alec S. Ren, Malvern, PA (US)

(21) Appl. No.: 13/697,450

(22) PCT Filed: May 16, 2011
(86) PCT No.: PCT/US2011/036675

§ 371 Date: Nov. 12, 2012

Related U.S. Application Data
(60) Provisional application No. 61/345,149, filed on May 16, 2010.
(60) Provisional application No. 61/345,181, filed on May 17, 2010.

(51) Int. Cl. C12Q 1/70; C12Q 1/68

(52) U.S. Cl. 435/5; 435/6.11; 435/6.12

(57) ABSTRACT

The present application provides a method of monitoring patients of chronic hepatitis B virus (HBV) infection undergoing nucleoside/nucleotide analogue antiviral treatment for treatment efficacy and for risk of drug-resistance, by simultaneous determination of quantities of viral DNA and identification of mutant viruses responsible for drug-resistance. This invention also provides methods and reagents for highly sensitive identification/quantification of KRas oncogene mutations from body fluids or tumor tissues and the use of these methods for cancer risk assessment, cancer early detection, treatment outcome prediction, and treatment monitoring.

### RELATED APPLICATIONS

This application claims the benefit of the filing date of U.S. Provisional Application No. 61/345,149 which was filed on May 16, 2010 and U.S. Provisional Application No. 61/345,181, which was filed on May 17, 2010. For the purpose of any U.S. application or patent that claims the benefit of U.S. Provisional Application No. 61/345,149 or U.S. Provisional Application No. 61/345,181, the content of these earlier filed applications is hereby incorporated by reference in their entirety.

### TECHNICAL FIELD

The present application relates to mutations of human hepatitis B virus, mutations of human KRas oncogene, and methods for genetic mutation detection and quantification. This application also relates to methods and test kits for treatment monitoring, for disease early detection and/or risk assessment screening, and for prognosis.

### BACKGROUND

Chronic infection by hepatitis B virus (HBV) is one of the major health burdens worldwide. Without effective intervention, as many as a quarter of the chronically infected individuals may develop liver cirrhosis after repeated liver injuries over a course of several decades; a portion of these patients will develop liver cancer (Liaw, Y. F. and C. M. Chu, Hepatitis B virus infection. Lancet, 2009. 373(9663): p. 582-92; Liang, T. J., Hepatitis B: the virus and disease. Hepatology, 2009. 49(5 Suppl): p. S13-21, both incorporated by reference). A large body of evidence, highlighted by the Risk Evaluation Viral Load Elevation and Associated Liver Disease (REVEAL) study, has demonstrated that active viral replication, indicated by a serum HBV DNA level>10⁴ copies per ml, plays a critical role in the disease progression to cirrhosis and liver cancer (Chen, C. J., et al., Risk of hepatocellular carcinoma across a biological gradient of serum hepatitis B virus DNA level. JAMA, 2006. 295(1): p. 65-73; Iloeje, U. H., et al., Predicting cirrhosis risk based on the level of circulating hepatitis B viral load. Gastroenterology, 2006. 130(3): p. 678-86, both incorporated by reference). Accordingly, the goal of treatment is now focused on the suppression of viral replication (Liaw, Y. F., et al., Acute exacerbation and hepatitis B virus clearance after emergence of YMDD motif mutation during lamivudine therapy. Hepatology, 1999. 30(2): p. 567-72, incorporated by reference). Since the viral particles propagated in the liver are directly released into the blood, the way to monitor treatment efficacy is to measure the level of viral DNA in the bloodstream.

Five orally administered anti-HBV agents have been approved in USA as mono- or combination therapies for chronic hepatitis B. They are lamivudine, telbivudine and entecavir that belong to nucleoside analogues, and adefovir and Tenofovir that are nucleotide analogues. These nucleos(t)ide analogues (NAs) are convenient to use, effective in suppressing viral replication, and very well tolerated, compared with the interferon therapy. Suppression of viral replication with NAs is accompanied by normalization of alanine transaminase levels, histological improvement manifested by reversion of liver fibrosis/cirrhosis, and reduced incidence of HCC (Liaw, Y. F., et al., Lamivudine for patients with chronic hepatitis B and advanced liver disease. N Engl J Med, 2004. 351(15): p. 1521-31, incorporated by reference). On the other hand, long term treatment is needed in most patients because the viral genome is stably maintained in the infected hepatocytes and not directly targeted by the NAs (Moraleda, G., et al., Lack of effect of antiviral therapy in nondividing hepatocyte cultures on the closed circular DNA of woodchuck hepatitis virus. J Virol, 1997. 71(12): p. 9392-9, incorporated by reference). However, long term treatment is, to various degrees, associated with the selection/development of drug resistant mutant viruses which can lead to acute exacerbation or hepatic decompensation, if a rescue therapy is not initiated in time (Liaw, Y. F., et al., Acute exacerbation and hepatitis B virus clearance after emergence of YMDD motif mutation during lamivudine therapy. Hepatology, 1999. 30(2): p. 567-72; Lok, A. S., et al., Long-term safety of lamivudine treatment in patients with chronic hepatitis B. Gastroenterology, 2003. 125(6): p. 1714-22, both incorporated by reference). Therefore, early detection or prediction of drug resistance is important in the management of chronic hepatitis B, especially in high risk patients such as those with cirrhosis.

Taken together, effective monitoring of treatment requires (1) measuring levels of viral DNA in the blood, and (2) early detection of the potential drug-resistant mutant viruses. Clinically, measuring the amount of viral DNA is done by using a TaqMan real time PCR method which cannot measure mutations at the same time, while the mutations are currently detected using DNA sequencing or the Line Probe assay (LiPA), neither of them can measure the amount of viral DNA.

Resistance to lamivudine, telbivudine and entecavir all involve mutations at the reverse transcriptase gene codon 204 (rt204), which codes for a methionine (M) at the so-called “YMDD motif”. A real time PCR based assay can measure the amount of WT viral DNA and mutant viral DNA at the same time so that early detection of drug resistance can be achieved cost-effectively. Such assays have been reported in the past few years (Chieochansin, T., et al., Rapid detection of lamivudine-resistant hepatitis B virus mutations by PCR-based methods. Tohoku J Exp Med, 2006. 210(1): p. 67-78; Yoshida, S., et al., Quantification of lamivudine-resistant hepatitis B virus mutants by type-specific TaqMan minor groove binder probe assay in patients with chronic hepatitis B Ann Clin Biochem, 2008. 45(Pt 1): p. 59-64; Shih, Y. H., et al., Hepatitis B virus quantification and detection of YMDD mutants in a single reaction by real-time PCR and annealing curve analysis. Antivir Ther, 2008. 13(4): p. 469-80, all incorporated by reference). However, none of these assays can reliably detect rt204 mutations. This is because the viral genomes show high levels of polymorphism (variations) among different patients; a regular probe of 15-20 nucleotides in length is likely to encounter polymorphisms, causing erroneous readout in both quantification and melting curve analysis.

KRas oncogene codes for KRas oncoprotein which plays an important role in promoting cell growth. KRas protein activity is tightly regulated, being turned “on” and “off” at specific times. If the KRas protein is constantly turned on, cell growth will become uncontrollable, and a tumor will develop. Mutations at the codon 12 and codon 13 of the KRas gene can cause constitutive activation of the KRas protein, i.e., the KRas protein is now locked in an “always on” state (Yamamoto, F. and M. Perucho, Activation of a human c-K-ras oncogene. Nucleic Acids Res, 1984. 12(23): p. 8873-85, incorporated by reference). KRas codon 12/13 mutations have been attributed to as at least one of the causative factors of human cancers, and have been detected frequently in pancreatic cancer, colorectal cancer (CRC) and certain other cancers (Bos, J. L., ras oncogenes in human cancer: a review. Cancer Res, 1989. 49(17): p. 4682-9, incorporated by reference).

Mutant KRas DNAs in the tumor cells can be released into the blood circulation when the tumor cells die. Detection of the KRas mutations from the circulation may, therefore, facilitate the early detection of cancer. It will also be helpful to monitor the level of KRas mutation before and after the surgical removal of the tumor to see if there is a hidden tumor or if the tumor is coming back (Diehl, F., et al., Circulating mutant DNA to assess tumor dynamics. Nat Med, 2008. 14(9): p. 985-90, incorporated by reference). For these purposes, it is necessary to quantify the KRas mutants with very high sensitivity.

Colorectal cancer (CRC) is the second most prevalent cancer and the second leading cause of cancer death in the US. About 40-50% of CRC tissues have detectable KRas mutations at the codons 12 and 13 which are responsible for malignancy and unresponsiveness to certain chemotherapies. The KRas mutation status in tumor tissues is now routinely determined to guide the selection of appropriate chemotherapies. The methods include DNA sequencing, real time PCR, and high resolution melting analysis. These currently available methods require the mutants to constitute more than 5% of “total” KRas gene. In addition, these methods are qualitative (positive or negative), not quantitative. Therefore, these methods cannot be used for the purpose of early detection or post-surgery monitoring. In addition, the currently used real time PCR test for the KRas codon 12/13 mutations uses 7 probes in 7 separate reactions to detect 7 most common mutations. The less common mutations will likely be undetectable.

Polymerase chain reaction (PCR) remains to be the most commonly used method for mutation detection because it can amplify and detect minute amount of target DNA. A minimum of 10 copies of mutants can be detected under the optimal conditions, in the absence of the wild type (WT), non-mutated, DNA. However, the mutant will be undetectable in the presence of more than 20-fold excess of the WT DNA without additional technologies. In the blood circulation, due to physiological turnover of the normal cells (such as the white blood cells), the WT DNA can be in more than 100 fold excess of the mutant DNA for any particular gene. Thus, detecting the tumor-derived DNA mutation from blood (or other body fluids such as urine) is technically challenging.

In this invention, an oligonucleotide is developed to inhibit the amplification of the WT KRas DNA thereby increasing the relative ratio of the mutant and the mutant detection sensitivity. This WT inhibitory oligonucleotide, or WT PCR blocker, is comprised of a modified nucleotide such as the locked nucleic acid (LNA). LNA is a nucleotide analogue that has increased specificity and affinity toward the complementary nucleotide (Singh, S. K., et al., LNA (locked nucleic acids): synthesis and high-affinity nucleic acid recognition. Chem Commun, 1998. 1998(4): p. 455-456; Hertoghs, K. M., J. H. Ellis, and I. R. Catchpole, Use of locked nucleic acid oligonucleotides to add functionality to plasmid DNA. Nucleic Acids Res, 2003. 31(20): p. 5817-30, both incorporated by reference). Oligonucleotides incorporated with LNAs have been used to increase the mutant detection sensitivity by inhibiting the amplification of the WT non-mutated DNA (Nagai, Y., et al., Genetic heterogeneity of the epidermal growth factor receptor in non-small cell lung cancer cell lines revealed by a rapid and sensitive detection system, the peptide nucleic acid-locked nucleic acid PCR clamp. Cancer Res, 2005. 65(16): p. 7276-82; Laughlin, T. S., et al., Rapid method for detection of mutations in the nucleophosmin gene in acute myeloid leukemia. J Mol Diagn, 2008. 10(4): p. 338-45, both incorporated by reference). However, such reports are infrequent because it is often difficult to develop such LNA-containing oligonucleotide to effectively suppress the amplification of the wild-type DNA while not affecting the amplification of the mutant DNA.

### SUMMARY

Because the viral genomes show high levels of polymorphism (variations) among different patients, a regular probe of 15-20 nucleotides in length is likely to encounter polymorphisms, causing erroneous readout in both quantification and melting curve analysis. Therefore it is necessary to design a probe that is as short as possible. In addition, the rt204 codon has at least five variants which include ATG (WT), ATT, ATC, ATA and GTG. The above mentioned assays were not designed to identify the ATA and ATC variants. In this invention, the test was redesigned to have significantly shorter probes. For the first time, all five variants of HBV codon rt204, including ATA and ATC, can be detected reliably using a real time PCR-based test. As a result, this improved real time PCR test is now suitable for use in clinical settings.

This invention presents a novel single test for HBV viral DNA quantification and drug resistant mutant virus detection for use in patients undergoing long term treatment using nucleotide/nucleoside analogues. The test is developed using a primer-probe partial overlap approach such that the effective length of the probe is only a few nucleotides, thereby making the test less prone to errors caused by the HBV genome polymorphism. The use of this test allows reliable detection of HBV mutations and cost-effective monitoring of treatment. The inventor has found that by keeping the probe as short as possible detection of polymorphisms is minimized, while at the same time, permitting efficient detection of mutations encoding the rt204 codon its many variants, for example ATG (WT), ATT, ATC, ATA and GTG.

In addition, this invention also presents two highly sensitive assay platforms for the detection of KRas codon 12/13 mutations. One is a qualitative PCR-based mutation detection system exemplified by DNA sequencing; the other is a quantitative mutant detection system exemplified by a 2-stage real time PCR. Both methods relied on the use of a wild-type PCR blocker that can efficiently suppress the amplification of WT KRas DNA without affecting the amplification of the KRas mutants.

This invention presents a WT PCR blocker which allows quantification of KRas mutants in the presence of more than 10,000-fold excess of the wild type KRas DNA, together with general rules for designing such WT PCR blockers for the detection of other gene mutations. This invention also includes a rationally designed PCR probe (KRas Probe #1) which allowed reliable identification of many variants of codon 12/13. Additional probes are being developed for use with the Probe #1 either together in one reaction or in separate reactions.

### BRIEF DESCRIPTION OF THE FIGURES

FIG. 1. Short probe designs.

FIG. 2. Primer-probe partial overlap simplifies melting patterns.

FIG. 3. Quantification of DNA and identification of mutations in a single test.

FIG. 4. Use of a single test to monitor patients undergoing lamivudine treatment.

FIG. 5. Suppression of amplification of WT DNA by WT PCR blocker and differential denaturing.

FIG. 6. KRas codon 12/13 real time PCR and use in clinical samples.

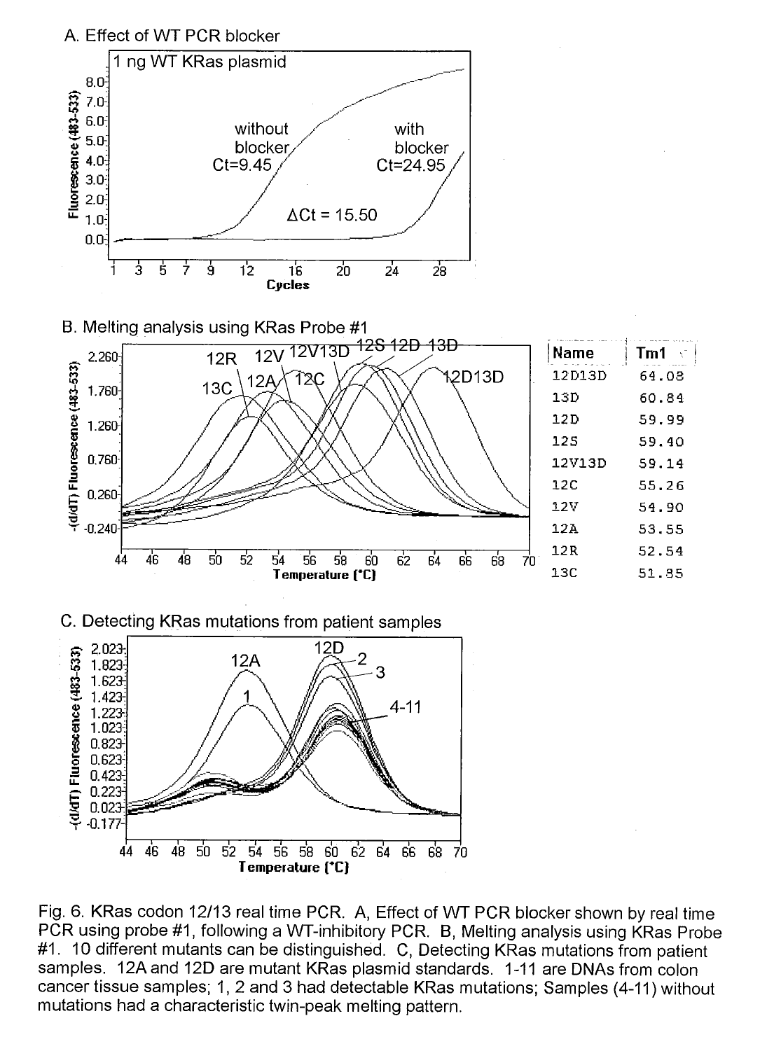

### DETAILED DESCRIPTION OF THE INVENTION

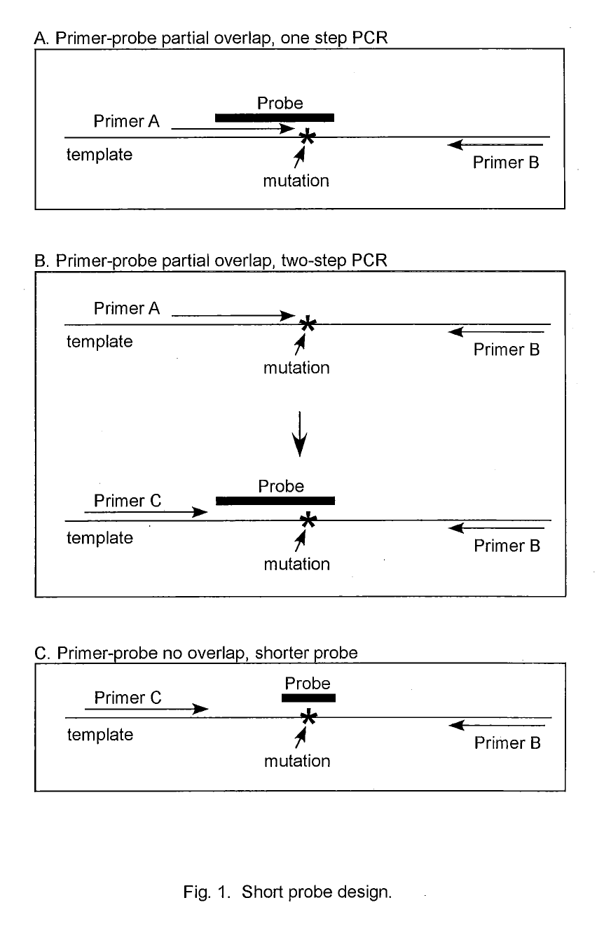

Referring to FIG. 1, there is shown the probe spans the region of DNA sequence where mutation of interest occurs. This probe can be any form of real time PCR probe such as SimpleProbe, molecular beacon, or TaqMan probe. The hybridization probes (or FRET probes) may also be used when the sensor probe is in overlap with the Primer A, but it is necessary to make sure the anchor probe is not affected by the potential nucleotide variations. The probe has more than 70% sequence identity to the targeted wild type (WT) sequence or its complementary strand. The mismatch or mismatches in the probe are designed to create better differentiation between the mutants and the WT, and among different mutants. In certain embodiments, the probe sequence may be identical to the wild type sequence, if enough differentiation between the WT and the mutants and among the mutants can be achieved.

In further detail, still referring to FIG. 1, one of the PCR amplification primers partially overlaps with the probe, and is in the same direction as the probe. This primer is designated as the Primer A (“a first primer”) for the convenience of description. The other amplification primer is designated as the Primer B (“a second primer”). The Primer A does not cover the mutation site, and thus the mutations can be detected by the probe during the amplification as well as after the amplification (by melting curve analysis). Primer A can have up to 50 nucleotides in length, and may have degenerated nucleotides. However, such degenerated nucleotides are not in overlap with the probe.

In further detail, referring to FIG. 1B, a two-step PCR may be used in which Primer A, partially overlapping with the probe, is used in step 1 PCR in the absence of the probe. Primer C (“a third primer”), overlapping with Primer A but not with the probe, is used in the step 2 real time PCR reaction in the presence of the probe for better PCR signals. In some embodiments, Primer B can also be “a fourth primer”. The two-step PCR allows the use of TaqMan probe for quantification.

Referring now to FIG. 1C, a short probe of 5-10 nucleotides long can be used in real time PCR with or without overlap with a primer. However, because the probe is too short to generate amplification signals, amplification will have to be dependent on a DNA-binding dye, such as SYBR green. Thus, the PCR is carried out with symmetric or asymmetric concentrations of Primer C and Primer B. SYBR Green signals are used for amplification purpose. The probe, which can be of any format, is used for melting curve analysis. However, the fluorescence channel of the probe must be distinguishable from the signals of SYBR Green. Because of this, a SimpleProbe or probes labeled with 6-FAM or fluorescein cannot be used in conjunction with SYBR Green.

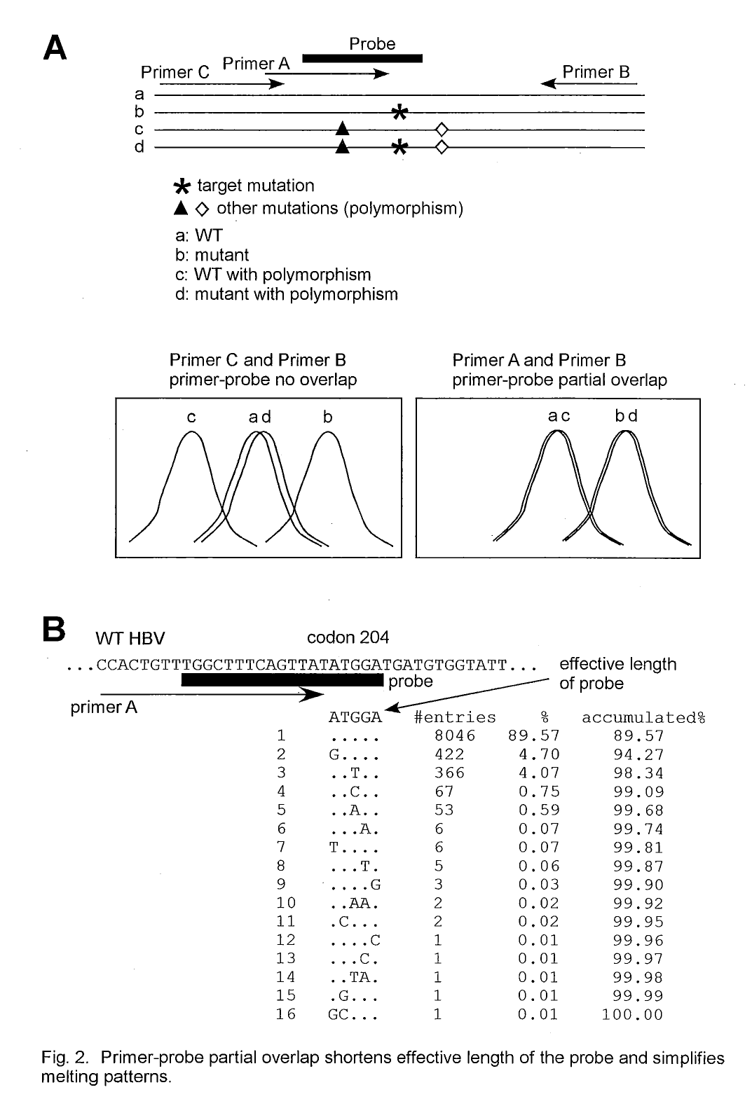

Referring to FIG. 2, PCR amplification using Primer A (and Primer B) will eliminate any potential polymorphism for the probe. Thus, the effective length of the probe, which is the part of the probe that responds to the mutations/mismatches, is the part of the probe that is not in overlap with the Primer A. The part of the probe that is in overlap with the Primer A functions to increase the melting temperature of the probe so that the probe has a high enough melting/annealing temperature to be used for quantification. Therefore, the short probe design, most effectively by the primer-probe partial overlap approach, simplifies the melting curve patterns, making the test results more applicable to read and more reliable. An example of simplified sequence patterns is shown in FIG. 2B. 8983 HBV sequences were retrieved from GenBank by BLAST. The sequence patterns corresponding to the effective probe length was sorted based on their frequencies in GenBank. The first 5 patterns are the WT (ATG at codon 204), GTG, ATT, ATC and ATA; they account for more than 99% of all GenBank HBV sequences. Thus, it is clear that the short effective probe length (5 nucleotide-long) made it possible to correctly identify more than 99% of the HBV sequences in the GenBank.

The HBV wild type sequence encoding the polymerase (reverse transcriptase) gene is highly polymorphic. Exemplary sequences include GenBank entries: AB55402, JF439940, GU456642, and HM358328. Exemplary HBV primer sequences are listed below:

HBV Primer A
5′-ttcccccactgtttggctttcagttat-3′
HBV primer B
5′-atgacgtcacagacttggcccccaatac-3′
HBV Probe #1
5′-[6FAM]-tggctttcagttaTGTTGa-[BHQ1]
HBV Probe #2
Cy5-tcagttataTAGa-IowaBlack
HBV Probe #3
HEX-ttggctttcagttatatcga-BBQ
Capital letters indicate LNA nucleotides.

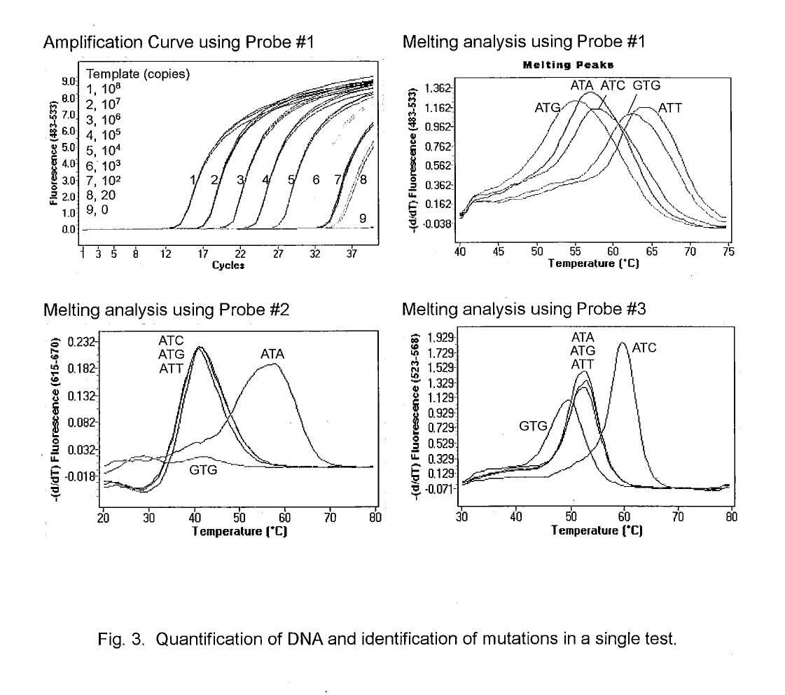

PCR can be carried out with symmetric or asymmetric concentrations of Primer A and Primer B. In the asymmetric PCR, the concentration of Primer A is 2-100 fold less than that of the Primer B, but typically 5-10 fold less than that of Primer B. The concentration of Primer A is at least 0.01 micro molar, and is typically used at 0.1 micro molar. The use of excessive Primer B allows excessive production of the single stranded of DNA that can be bound by the probe, thereby enhancing the probe signals in the amplification curve (FIG. 3). This also allows melting curve analysis after the amplification (FIG. 3).

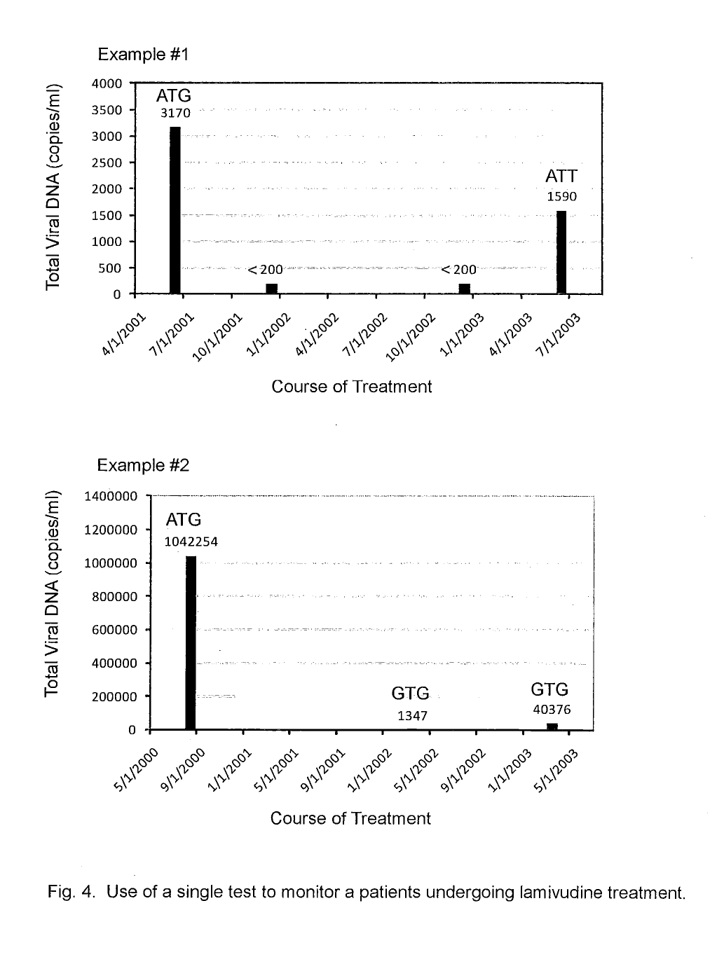

Referring to FIG. 4, two patients of chronic hepatitis B undergoing lamivudine treatment were “monitored” retrospectively, using the single PCR test in this invention. As shown in FIG. 4, both patients initially expressed wild type codon rt204 (“ATG”) but over time as treatment progressed, rt204 mutated to drug resistant forms, e.g., (“ATT”) and (“GTG”). Both viral titer and mutant viruses can be effectively determined using a single test.

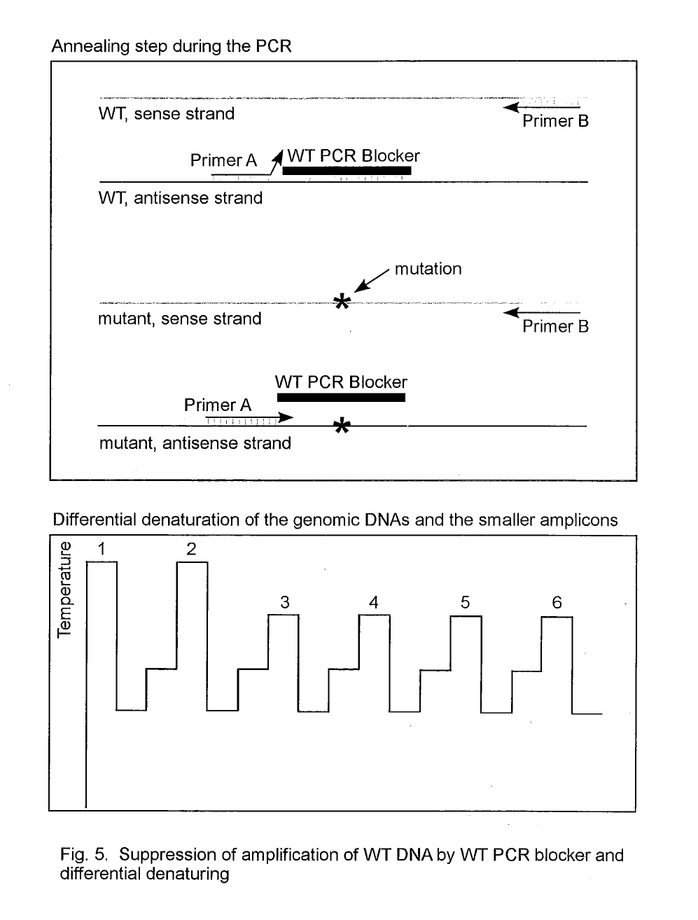

Referring now to FIG. 5, there is shown the WT PCR blocker spans the region of DNA sequence where mutation of interest occurs. At the mutation site, the WT PCR blocker has a perfect match to the WT KRas sequence or the complementary strand of the WT KRas sequence, and has a mismatch or mismatches to the mutant DNA sequence due to the presence of mutation(s). The PCR blocker has up to 50 nucleotides, and contains at least one LNA nucleotide. The WT PCR blocker is phosphorylated at the 3′-end so that it will not function as a primer.

Exemplary KRas cDNA sequences can be found in GenBank accession NM_{—}004985. KRas genomic DNA sequence can be found in GenBank accession NT_{—}009714.17. Exemplary primer sequences include:

KRas WT PCR blocker
5′-gcctacgCcaCCagctc-PH
KRas Primer A
5′-gtcaaggcactcttgcctacg-3′
KRas Primer B
5′-ggacgtccgtcacattttcattatttttattataaggc-3′
KRas primer A2
5′-tcaaggcactcttgcctacg-3 ′
KRas Probe #1
[6FAM]tgcctacgtcattagctccaac[BHQ1]
Capital letters indicate LNA nucleotides.
“-PH” stands for 3′-end phosphorylation.

In further detail, still referring to the invention of FIG. 5, one of the PCR amplification primers partially overlaps with the WT PCR blocker, and is in the same direction as the PCR blocker. This primer is designated as the Primer A for the convenience of description. The other amplification primer is designated as the Primer B. The Primer A in this invention does not cover the mutation site thus will allow the mutation to be amplified and be detected by a downstream application such as DNA sequencing or real time PCR. Primer A can have up to 50 nucleotides with more than 80% sequence identity to the target sequence. The overlap between the Primer A and the WT PCR blocker is at least one nucleotide. Primer A and Primer B may or may not contain LNA nucleotides. During thermal cycling, the PCR blocker will be able to bind to the WT sequence strongly due to the presence of the LNA nucleotide(s), but bind to the mutant sequence weakly due to the mismatch(es). This results in displacement of the Primer A on the wild type template sequence, but not so on the mutant DNA template. This results in inhibition of PCR amplification of the wild type KRas DNA, but allows amplification of the mutant KRas DNA.

In further detail, still referring to the invention of FIG. 5, PCR can be programmed to denature the DNA template (usually genomic DNA) at 95° C. for less than 10 cycles, followed by continued amplification at a lower denaturing temperature to denature just the amplicon obtained from primer A and primer B. This significantly reduces the synthesis of the WT antisense strands which is not suppressed by the WT PCR blocker, thereby causing stronger inhibition of amplification of the WT DNA.

The ultra-sensitive wild type-inhibitory direct DNA sequencing method for KRas mutation detection is comprised of a wild-type inhibitory PCR (Wi-PCR) followed by DNA sequencing. The Wi-PCR is performed by adding the WT PCR blocker to the otherwise regular PCR reaction that contains the amplification primers (Primers A and B), DNA polymerase, the polymerase buffer, dNTPs and the DNA template. The concentration of the Primer A and B can be in the range of 0.1 to 1 μM. The concentration of the Wi-oligo can be in the range of 0.2 to 50 μM. The Wi-PCR is performed for 25-55 cycles until sufficient amount of DNA is generated. The PCR product is purified to remove the free primers, and is subjected to DNA sequencing using the Primer B. To distinguish from the regular direct DNA sequencing, we name it Wi-direct DNA sequencing for wild type inhibitory direct DNA sequencing.

The Wi-PCR can be followed by any other mutation detection methods, in addition to direct sequencing. These methods can be either qualitative or quantitative, and the initial Wi-PCR can significantly increase their mutation detection sensitivity. These methods may include, but not limited to, solid phase hybridization (for example, Southern blotting and dot blotting), liquid phase hybridization (such as melting curve analysis), reverse hybridization (labeled PCR products hybridizing to the immobilized oligonucleotides), mass spectrometer, and real time PCR.

The ultra-sensitive quantitative KRas mutation detection system is comprised of a Wi-PCR followed by a real time PCR using a fluorescence-labeled oligonucleotide probe. This real time PCR can be, but not limited to, a TaqMan PCR using a hydrolysis probe, a FRET PCR, a SimpleProbe PCR, a Scorpion probe PCR, or a molecular beacon real time PCR. The Wi-PCR is performed for 10-20 cycles, followed by 30-40 cycles of real time PCR. This is designated as Wi-quantitative PCR or Wi-qPCR.

A “TaqMan” hydrolysis probe for KRas codon 12/13 mutations was developed for use in a non-hydrolysis asymmetric real time PCR. The probe was designed such that it can distinguish 11 known variants (including the wild type KRas sequence) in the melting curve analysis.

Provided herein is a method for evaluating therapy with an anti-hepatitis B virus (HBV) agent for treatment of a subject who has or who is likely to have an HBV infection, the method comprising: (a) providing a nucleic acid sample from the subject; (b) determining the identity of the codon at position 204 of the open reading frame of the reverse transcriptase gene on either the coding or the non-coding strand; and (c) evaluating whether the subject should undergo therapy with the HBV agent. In some embodiments, the method further comprise identifying a subject having an HBV infection. The anti-HBV agent can be a nucleoside or nucleotide analogue. The nucleoside analogue can be lamivudine, telbivudine or entecavir. The nucleotide analogue can be adefovir or Tenofovir. Also provided is a method of identifying an rt 204 mutation in HBV reverse transcriptase in a subject, the method comprising: (a) providing a nucleic acid sample from the subject; (b) contacting the nucleic acid with a first and second primer, wherein the first primer hybridizes to a nucleotide sequence that is proximal to a sequence encoding the rt204 codon and the second primer hybridizes to a nucleotide sequence that is distal to a sequence encoding the rt204 codon and one or more detectably labeled probes that hybridize to a sequence encoding the mutant rt204 codon to form a mixture; and (c) amplifying the nucleic acid. The method can further comprise analyzing the melting curves of the amplified nucleic acids. In some embodiments, the detectably labeled probe hybridizes to a sequence encoding the mutant rt204 codon or to a complementary strand and to a portion of the sequence recognized by the first primer. Each detectably labeled probe can comprise a different label. The label can be 6FAM, Cy5 or HEX. The nucleotide sequence encoding rt204 in the one or more detectably labeled probes can comprise the sequence ATG, ATT, ATC, ATA, or GTG. In some embodiments, the detectably labeled probe is less than about 10 nucleotides in length. Also provided is a method of identifying an rt204 mutation in HBV reverse transcriptase in a subject, the method comprising: (a) providing a nucleic acid sample from a subject; (b) contacting the nucleic acid with a first and second primer, wherein the first primer hybridizes to a nucleotide sequence that is proximal to a sequence encoding the rt204 codon and the second primer hybridizes to a nucleotide sequence that is distal to a sequence encoding the rt204 codon to form a mixture; and (c) amplifying the nucleic acid; and (d) contacting the amplified nucleic acid with a third and fourth primer, wherein the third primer hybridizes to a sequence that is proximal to a sequence encoding the rt204 codon and the fourth primer hybridizes to a nucleotide sequence that is distal to a sequence encoding the rt204 codon and a one or more detectably labeled probes that hybridize to a sequence encoding the mutant rt204 codon, wherein the sequence encoding the rt204 codon does not include the sequence recognized by the third primer to form a mixture; and (e) further amplifying the nucleic acid. The sequences of the second primer and the fourth primer can be the same. The method can further comprise analyzing the melting curves of the amplified nucleic acids. In some embodiments, the detectably labeled probe hybridizes to a sequence encoding the mutant rt204 codon and to a portion of the sequence recognized by the first primer. Each detectably labeled probe can comprise a different label. The label can be 6FAM, Cy5 or HEX. The nucleotide sequence encoding rt204 in the one or more detectably labeled probes can comprise the sequence ATG, ATT, ATC, ATA, or GTG. In some embodiments, the detectably labeled probes is less than about 10 nucleotides. In some embodiments the amplified nulceic acid of step (c) is isolated from the mixture. The nucleic acid sample can be from a biological fluid or a tissue sample.

Also provided is a method for evaluating therapy with an anti-cancer agent for treatment of a subject, the method comprising (a) obtaining a nucleic acid sample from the subject; (b) determining the identity of the codon at position 12 or 13 of the open reading frame of the KRAS protooncongene; and (c) evaluating whether the subject should undergo therapy with the anti-cancer agent. The method can further comprise identifying a subject who has or who is likely to have a cancer with a mutated KRAS protooncogene. The nucleic acid can be from a biological fluid sample or a biopsy sample. The anticancer agent can be cetuximab or panitumumab. The cancer can be any cancer comprising an KRAS mutation at position 12/13, for example, colorectal cancer, pancreatic cancer, lung cancer, or ovarian cancer. Also provided is a method of identifying a mutation in the KRAS protooncogene in a subject, the method comprising: (a) providing a nucleic acid sample from a subject; (b) contacting the nucleic acid with a first and second primer, wherein the first primer hybridizes to a nucleotide sequence that is proximal to a sequence encoding codons 12 or 13 and the second primer hybridizes to a nucleotide sequence that is distal to a sequence encoding codons 12 or 13 and a wild type PCR blocking oligonucleotide that hybridizes to a sequence encoding codons 12 or 13 or a sequence complementary to a sequence encoding codons 12 or 13 to form a mixture; and (c) amplifying the nucleic acid. The wild type PCR blocking oligonucleotide can comprise one or more locked nucleic acids (LNA). The wild type PCR blocking oligonucleotide hybridizes to a sequence encoding codons 12 or 13 and to a portion of the sequence recognized by the first primer. The mixture can further comprise one or more detectably labeled nucleotide probes, wherein the nucleotide hybridizes to a sequence encoding codons 12 or 13 or a sequence complementary to a sequence encoding codons 12 or 13. Also provided is a method for clinical management a subject who has cancer, the method comprising (a) obtaining a nucleic acid sample from the subject; (b) determining the identity of the codon at position 12 or 13 of the open reading frame of the KRAS protooncongene; and (c) evaluating whether the cancer has relapsed.

In some embodiments, the WT PCR blocker partially overlaps with primer A which is the primer in the same direction as the WT PCR blocker. The WT PCR blocker can have a melting temperature (Tm) with the WT DNA [Tm(blocker/WT)] higher than 70° C. but lower than 90° C. In some embodiments, the WT PCR blocker has a Tm(blocker/WT) at least 4° C. higher than its melting temperature with the mutant DNA [Tm(blocker/mutant)]. In some embodiments, primer A has a melting temperature lower than Tm(blocker/WT) but equal to or slightly higher or lower than Tm(blocker/mutant). In some embodiments, amplicon of primer A and primer B is selectively denatured in the first step PCR reaction to enhance suppression of the amplification of WT DNA.

### EXAMPLES

### Example 1

quantitative PCR for the HBV rt204 codon. The probe and the amplification primers A and B were 5′-[6FAM]-tggctttcagttaTGTTGa-[BHQ1], 5′-ttcccccactgtttggctttcagttat-3′, and 5′-atgacgtcacagacttggcccccaatac-3′, respectively. Capital letters indicate locked nucleic acids. The PCR was carried out using the Genotyping Master Mix (Roche), 0.1 μM Primer A, 0.5 μM Primer B, 0.1 μM of the probe, and the template DNA. The thermal profile was 95° C. for 10 min to activate the polymerase, followed by 40 cycles of 95° C. 10 seconds, 55° C. 10 seconds with fluorescence requisition, and 72° C. 1 second. Immediately after the amplification, a melting curve analysis was performed during a linear temperature increase from 40 to 75° C. For quantification purpose, serial diluted plasmids carrying rt204(ATG) were included so that a concentration standard curve can be generated. To distinguish which rt204 variant was in the sample, plasmids carrying different variants were included in the experiment to generate melting standards.

### Example 2

quantitative PCR for the HBV rt204 codon. The amplification primers A and B were 5′-ttcccccactgtttggctttcagttat-3′, and 5′-atgacgtcacagacttggcccccaatac-3′, respectively. Three probes were used in a single reaction to enhance the distinction among the five variants; they are Probe #1 (5′-[6FAM]-tggctttcagttaTGTTGa-[BHQ1]), Probe #2 (Cy5-tcagttataTAGa-IowaBlack) and Probe #3 (HEX-ttggctttcagttatatcga-BBQ). Capital letters indicate locked nucleic acids. The PCR was carried out using the Genotyping Master Mix (Roche), 0.1 μM Primer A, 0.5 μM Primer B, 0.1 μM for each probe, and the template DNA. The thermal profile was 95° C. for 10 min to activate the polymerase, followed by 40 cycles of 95° C. 10 seconds, 50° C. 10 seconds with fluorescence requisition, and 72° C. 1 second. Immediately after the amplification, a melting curve analysis was performed during a linear temperature increase from 20 to 75° C. For quantification of patient samples, plasmids carrying rt204(ATG) were serial diluted with DNAs purified from normal human serum and used in PCR to generate a concentration standard curve. To distinguish which rt204 variant was in the sample, plasmids carrying different variants were included in the experiment to generate melting standards.

### Example 3

WT-inhibitory PCR for KRas codon 12/13 mutations. The WT PCR blocker, and the amplification primers A and B are 5′-gcctacgCcaCCagctc-PH, 5′-gtcaaggcactcttgcctacg-3′ and 5′-ggacgtccgtcacattttcattatttttattataaggc-3′, respectively. Capital letters indicate LNA nucleotides. “-PH” stands for 3′-end phosphorylation. The Wi-PCR was carried out using a hot-start Taq DNA polymerase in the appropriate PCR buffer, 200 μM dNTP, 0.5 μM each of the amplification primers, 2 μM of the WT PCR blocker, and the template DNA. The thermal profile was 95° C. for 2-10 min to activate the polymerase, followed by 20 cycles (for downstream qPCR) or 45 cycles (for DNA sequencing) of 95° C. 10 seconds, 76° C. 20 seconds, 60° C. 10 seconds and 65° C. 10 seconds. The thermal profile could also be 2 cycles of 95° C. 10 seconds, 60° C. 10 seconds, 68° C. 10 seconds, followed by 18 cycles of 84° C. 10 seconds, 60° C. 10 seconds, 68° C. 10 seconds.

### Example 4

### WT-inhibitory quantitative PCR for KRas codon 12/13 mutations. The Wi-PCR reaction performed for 20 cycles, as described above, was diluted by 32-fold with H2O or Tris/EDTA buffer. One microliter of the diluted PCR was added to a PCR reaction which contains Genotyping Master Mix (Roche), 5 mM MgCl2, 0.1 μM forward primer (5′-tcaaggcactcttgcctacg-3′), 0.5 μM reverse primer (5′-ggacgtccgtcacattttcattatttttattataaggc-3′), and 0.1 μM KRas Probe #1 (5′-[6FAM]tgcctacgtcattagctccaac[BHQ1]). Amplification was performed by 30 cycles of 84° C. 10 seconds, 57° C. 10 seconds, 68° C. 10 seconds, and 50° C. for 15 seconds with fluorescence detection. Immediately after the amplification, a melting curve analysis was performed at a temperature range of 25-75° C. To be able to quantify the amount of mutant, serial diluted plasmids carrying the mutant (the 12D variant) were included in the Wi-PCR and further amplified in the real time PCR. For the melting curve analysis, plasmids carrying different KRas sequences were included in the experiment for comparison purpose. In certain cases when the wild type DNA was not inhibited completely, the melting curve will show a mix of two peaks, one representing the mutant and the other wild type DNA. The amount of the mutant DNA will be estimated based upon the relative height of the two peaks. It should be noted that additional probes may be added to the same reaction or in separate reactions to improve differentiation among different KRas codon 12/13 variants, especially in between 12D and 12S, and between 12C and 12V.

### Example 5

### Quantification of KRas 12/13 mutants by SYBR Green real time PCR. The PCR was carried out as described in Example 3, with the addition of SYBR Green dye, for 50 cycles. To be able to quantify the amount of mutant, serial diluted plasmids carrying the mutant (the 12D variant) were included in the PCR. The samples that show positive amplification were then selected, diluted 1:32 with H2O or Tris/EDTA buffer, and subjected to a WT-inhibitory quantitative PCR described in Example 4 for a melting curve analysis. The plasmids carrying different KRas sequences were included as melting curve standards.

The advantages of the present invention include, without limitation, increased functionality without extra cost (semi-quantification of HBV drug resistant mutants in addition to viral load measurement), and increased accuracy due to the short effective length of the probe. In broad embodiment, the present invention can be applied to the detection of other HBV drug resistant mutants, and other genetic mutations in general. The use of TaqMan probe for melting curve analysis in asymmetric PCR using a 5′-3′ exo-minus Taq polymerase allows multiplex melting curve analysis at a much lower cost. The advantages of the present invention also include, without limitation, detection and quantification of KRas mutations with an extraordinarily high sensitivity. In broad embodiment, the present invention can be applied to the detection of other genetic mutations with ultra-high sensitivity.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

1. A method for evaluating therapy with an anti-hepatitis B virus (HBV) agent for treatment of a subject who has or who is likely to have an HBV infection, the method comprising:
(a) obtaining a nucleic acid sample from the subject;
(b) detecting one or more mutations of HBV reverse transcriptase gene and the total HBV viral load in a single test;
(c) evaluating whether the subject should undergo therapy with the HBV agent.

2-5. (canceled)

6. The method of identifying an rt204 mutation in HBV reverse transcriptase in a subject, the method comprising:
(a) obtaining a nucleic acid sample from the subject;
(b) contacting the nucleic acid with a first and second primer, wherein the first primer hybridizes to a nucleotide sequence that is proximal to a sequence encoding the rt204 codon and the second primer hybridizes to a nucleotide sequence that is distal to a sequence encoding the rt204 codon and one or more detectably labeled probes that hybridize to a sequence encoding the mutant rt204 codon to form a mixture; and
(c) amplifying the nucleic acid to quantify viral DNA and to determine mutation(s) at rt204 in a single test.

7. The method of claim 6, wherein determine mutation(s) is via the melting curves of the amplified nucleic acids.

8. The method of claim 6, wherein the detectably labeled probe hybridizes to a sequence encoding the mutant rt204 codon or to a complementary strand and to a portion of the sequence recognized by the first primer.

9-11. (canceled)

12. The method of claim 6, wherein the detectably labeled probe is less than about 10 nucleotides in effective length.

13-22. (canceled)

23. A method for evaluating therapy with an anti-cancer agent for treatment of a subject, the method comprising:
(a) obtaining a nucleic acid sample from the subject;
(b) determining the identity of the codon at position 12 and 13 of the open reading frame of the KRAS protooncongene, and the proportion of the mutated KRAS gene.
(c) evaluating whether the subject should undergo therapy with the anti-cancer agent.

24. The method of claim 23, further comprising identifying a subject who has or who is likely to have a cancer with a mutated KRAS protooncogene.

25. The method of claim 23 or 24, wherein the nucleic acid is from a biological fluid sample or a biopsy sample.

26. The method of claim 23, wherein the anticancer agent is cetuximab or panitumumab.

27. The method of claims 23 and 24, wherein the cancer comprises colorectal cancer, pancreatic cancer, lung cancer, or ovarian cancer.

28. A method of determining the identity of the codon at position 12 and 13 of the open reading frame of the KRAS protooncongene and the proportion of the mutated KRAS gene in a subject, the method comprising:
(a) obtaining a nucleic acid sample, from a subject;
(b) contacting the nucleic acid with a first and second primer, wherein the first primer hybridizes to a nucleotide sequence that is proximal to a sequence encoding codons 12 and 13 and the second primer hybridizes to a nucleotide sequence that is distal to a sequence encoding codons 12 and 13 and a wild type PCR blocking oligonucleotide that hybridizes to a sequence encoding codons 12 and 13 or a sequence complementary to a sequence encoding codons 12 and 13 to form a mixture; and
(c) amplifying the nucleic acid.

29. The method of claim 28, wherein the wild type PCR blocking oligonucleotide comprises one or more locked nucleic acids (LNA).

30. The method of claim 28 or 29, wherein the wild type PCR blocking oligonucleotide hybridizes to a sequence encoding codons 12 and 13 and to a portion of the sequence recognized by the first primer.

31. (canceled)

32. A method for clinical management a subject who has cancer, the method comprising
(a) obtaining a nucleic acid sample from the subject;
(b) determining the identity of the codon at position 12 and 13 of the open reading frame of the KRAS protooncongene; and determining the proportion of the mutated KRAS gene, and
(c) evaluating whether the cancer has relapsed.

33. The method of claim 1, wherein said one or more mutations of HBV reverse transcriptase, gene are those associated with resistance to the anti-HBV agent that is in use, or plan to be used, by the subject.

34. The method of claim 1 wherein said single test is PCR-based method involving melting curve analysis.

35. The method of claim 6 wherein said one or more detectably labeled probes are able to identify ATA, ATC, ATG, ATT and GTG variants of rt204.

36. The method of claim 28, further comprising, following step (c), contacting the amplified nucleic acid with a third and fourth primer, wherein the third primer hybridizes to a sequence that is proximal to a sequence encoding codons 12 and 13, and the fourth primer hybridizes to a nucleotide sequence that is distal to a sequence encoding codons 12 and 13, and one or more detectably labeled probes that hybridize to a sequence encoding codons 12 and 13 to form a mixture; and further amplifying the nucleic acid.

37. The method of claim 36 wherein the sequences of the third primer can be different from or the same as the first primer in claim 28, and the sequences of the fourth primer can be different from or the same as the second primer in claim 28.

38. The method of claim 36, further comprising melting curve analysis of the amplified nucleic acids.

39. The method of claim 36, wherein the detectably labeled probes contain the wild type and/or mutated form of the coding sequence or its complementary sequence of KRAS codon 12 and 13.

40. The method of claim 28, further comprising, following step (c), a DNA sequence determination method from a group consisting of DNA sequencing, hybridization, restriction enzyme analysis, mass spectrometry, and real time PCR.

41. A method of inhibiting PCR amplification of a variant DNA, the method comprising:
(a) contacting the nucleic acid with a first and second primer, wherein the first primer hybridizes to a nucleotide sequence distal to the sequence of the target variation or mutation site and the second primer hybridizes to the sequence distal to the target variation or mutation site, and with a variant PCR blocking oligonucleotide that hybridizes to the variant at the target variation or mutation site to form a mixture; and
(b) amplifying the nucleic acid using a thermal program that allows selective denaturing of the amplicons.

42. The method of claim 41 wherein said variant can be the wild type or one of the mutants.

43. The method of claim 41 wherein said the variant-inhibitory oligonucleotide may contain one or more locked nucleic acids or peptide nucleic acids or any other special nucleotides that have increased binding affinity toward the complementary nucleotides.
